(19) United States
(12) Patent Application Publication
(10) Pub. No.: US 2025/0185983 A1
(43) Pub. Date: Jun. 12, 2025

(54) Determination of the Body Posture

(71) Applicant: Siemens Aktiengesellschaft, München (DE)

(72) Inventors: Raphaela Groten, Aachen (DE); Rebecca Johnson, München (DE)

(73) Assignee: Siemens Aktiengesellschaft, München (DE)

(21) Appl. No.: 18/842,171

(22) PCT Filed: Feb. 20, 2023
(86) PCT No.: PCT/EP2023/054180

§ 371 Date: Aug. 28, 2024

(30) Foreign Application Priority Data
Mar. 21, 2022 (DE) 10 2022 202 729.9

(51) Int. Cl. A61B 5/00

(52) U.S. Cl. CPC A61B 5/4561

(57) ABSTRACT

Various embodiments of the teachings herein include systems and methods for monitoring the posture of a user during use of an electronic device. An example includes: reading an image from a camera associated with the electronic device; determining a value representing the posture shown in the image; and transmitting a warning signal if the value falls in a warning range.

### CROSS-REFERENCE TO RELATED APPLICATIONS

This application is a U.S. National Stage Application of International Application No. PCT/EP2023/054180 filed Feb. 20, 2023, which designates the United States of America, and claims priority to DE Application No. 10 2022 202 729.9 filed Mar. 21, 2022, the contents of which are hereby incorporated by reference in their entirety.

### TECHNICAL FIELD

The present disclosure relates ergonomics. Various embodiments of the teachings herein include systems and/or methods for determining a user's posture while working on a PC.

### BACKGROUND

A general trend in the world of work is the transfer of all content to the digital world. Content is no longer handled in the form of paper or other hardware but rather on electronic devices such as tablets and PCs. This trend has been accelerated enormously by the pandemic since practically all over the world safety regulations such as for example distancing rules were put into practice by way of a shift to the “home office”. As a result, even more work is necessarily done on the PC. Even meetings and other personal communication which rather took place away from the PC before the pandemic are now carried out from the PC.

The proportion of seated activity has thus increased further. Prolonged seated activity is straining for the back and is blamed for some of the back problems increasingly present in the population. Reasons cited for this are for example physical inactivity and incorrect posture. Physical inactivity can be monitored by smartphones and smartwatches, for example in the form of counting steps. When it comes to posture, however, this is more difficult.

### SUMMARY

Teachings of the present disclosure include methods and arrangements with which feedback relating to posture can be given to a person while working on an electronic device, in particular a PC. For example, some embodiments include a method for determining the posture of a person (1) while working on an electronic device, in particular a PC, including: reading at least one image from a camera (3) of the electronic device, determining a value that represents the posture that can be seen in the image, and outputting a warning signal if the value is in a warning range.

In some embodiments, the value is determined from the relative apparent position of body reference points in the image.

In some embodiments, the body reference points are selected from: ears, jaw, shoulder, and sternum.

In some embodiments, the relative apparent position determined is a vertical distance between the reference points.

In some embodiments, the relative apparent position is determined for both sides of the body and a posture that deviates from the vertical is identified by way of a comparison.

In some embodiments, an altitude angle (5) at which the camera (3) is recording the person (1) is determined from the image and the position of the reference points is corrected using the altitude angle (5).

In some embodiments, the posture is determined for a sequence of images from the camera (3) and, out of the values determined in this way, those with a high probability of error are discarded.

In some embodiments, use is made of at least two cameras (3), in particular a 3D-capable camera.

As another example, some embodiments include an arrangement for determining the posture of a person (1) while working on an electronic device, in particular a PC, comprising: a camera (3) connected to the PC for image transmission purposes, and a program running on the PC for evaluating at least one image received from the camera, wherein the program is used to carry out one or more of the methods as described herein.

### BRIEF DESCRIPTION OF THE DRAWINGS

The teachings of the present disclosure are described and explained in more detail below on the basis of the exemplary embodiments illustrated in the figures. In the drawings:

FIG. 1 shows a person in a first, poor posture while working on a PC having a camera;

FIG. 2 shows an example method for determining the posture of the person incorporating teachings of the present disclosure;

FIG. 3 shows the person while working on a laptop having an integrated camera; and

FIG. 4 shows the person in a second, good posture while working on the PC.

### DETAILED DESCRIPTION

Some embodiments of the teachings herein include a method for determining the posture of a person while working on an electronic device, in particular a PC. In an example, at least one image from a camera of the electronic device is read. A value that represents the posture that can be seen in the image is then determined. Finally, a warning signal is output if the value is in a warning range.

Some embodiments include an arrangement for determining the posture of a person while working on an electronic device, in particular a PC. An example includes a camera connected to the electronic device for image transmission purposes and a program running on the electronic device for evaluating at least one image received from the camera, wherein the program is used to carry out one or more of the methods described herein.

As a result, the posture especially while working on a screen can be determined and monitored and the working person can be given a warning if the posture is not recommendable. The posture can thus be improved, provided that the person heeds the warning signal. In some embodiments, only means that are part of many working PCs are used in this case, namely the camera that is already integrated in laptops in most cases anyway. Even in the case of desktop PCs, even though a camera is often not integrated, there is often still one available due to the working conditions during the pandemic.

In some embodiments, the value can be determined from the relative apparent position of body reference points in the image. In this case, the body reference points are appropriate parts of the body which are selected such that they can be seen well in the image and their relative apparent position is a good indicator of the posture, especially the posture of the upper body. In this case, apparent denotes the position as presented in the two-dimensional image from the camera. Two points in the three-dimensional space whose connecting line is parallel to the camera viewing direction have no distance in the image from the camera, even if their three-dimensional distance is large. Conversely, two points whose connecting line is substantially perpendicular to the camera viewing direction have a distance that is proportional to the three-dimensional distance (and to the distance from the camera) in the image. In this case, relative denotes that the distance between two body reference points is considered, and not the absolute position of a body reference point.

Accurate posture analysis is not necessary at all; rather, it is enough to identify the essential element of a “bad” posture. Such a bad posture is usually seen as given when a sort of “hunched posture” is adopted, that is to say the back is bent toward the screen. Often, the shoulders are additionally raised by use of the mouse and keyboard. Both ensure that the body elements around the shoulder are more parallel to the camera than in the case of an upright and straight posture that is presumed to be “good”. In the case of the latter, the body elements around the shoulder are significantly more perpendicular to the camera viewing direction.

In the apparent position in the image, particular body reference points are therefore closer to one another when a posture defined as bad is adopted and further away from one another when a posture defined as good is adopted. Since absolute values for the apparent relative position depend very much on the person, some embodiments store these values, at least in part, over a longer period of time and determine the current posture from the stored values. For example, a maximum value for the distance between the body reference points can be determined from the stored values and this maximum value can be interpreted as a measure of a good posture. So that such a stored value is present at all, it can be advantageous to guide the person through a one-time configuration program during which different postures are adopted under instruction and the result of which is stored in the form of the camera image or values derived therefrom.

In some embodiments, the body reference points can be a selection from the following body parts: ears, jaw, shoulder, sternum. In particular, the distance between ears and shoulder, ears and sternum or jaw and sternum can be considered. These distances are a direct measure of the posture of the person and are relatively easy to identify in the image from the camera.

In particular, the shoulder and the ears can already be found if only the silhouette of the person is determined from the image. The silhouette is in turn relatively easy to find. It stands out well in particular in lighting and contrast differences in relation to the background that is significantly further away.

In addition to simple graphic methods, more complex methods of image evaluation can also be used. For example, a neural network can be used to perform image evaluation. Neural networks are known to deliver good image evaluation results if the images are complex or variable, meaning that conventional image recognition methods no longer work. Neural networks require training that is sufficiently variable and suitable for the application. This is expediently done using a sufficient number of images of different and differently dressed people, which images are taken over a range of possible image portions and possible camera positions (from above/from below). In this case, the range of the image portions is small since at least the head and shoulders, depending on the configuration also the upper sternum, must be able to be seen. Conversely, pure head shots are not enough to discern the posture and therefore also do not need to be considered and trained. The image elements that are to be discerned, that is to say the body reference points, are easy to determine manually as input values for the training.

The relative apparent position determined can be a vertical distance between the body reference points. In other words, the horizontal position of the body reference points is ignored since, for that part of the posture that is to be observed, only the inclination of the head, shoulders and upper sternum is relevant and this can be read purely from the vertical distance.

The relative apparent position can be determined for both sides of the body and a posture that deviates from the vertical can be identified by way of a comparison. Since the sides of the body are substantially symmetrical, it is possible to determine a slanted posture from a comparison of the body reference points between the left and right side. For example, a connection vector between the two positions of the ears and a connection vector between the positions of the shoulders can be defined. A number of conclusions can be drawn from these vectors.

If the direction of the vectors continues to differ from the horizontal over a relatively long time, the position of the camera may be slanted. This can be checked by evaluating other image elements. For example, straight lines in the image can be determined. These often run horizontally or vertically and therefore allow for a position check. Another possibility consists in the camera comprising a position sensor, as is the case in smartphones for example.

If the position of the camera is substantially horizontal, a slanted position of the vectors over a relatively long time may indicate a general slanted posture. This can also be detected and stored for the further evaluation. Conversely, in the case of a short-term slanted position, the determined posture values can be discarded since the person is just now changing their posture.

An altitude angle at which the camera is recording the person can be determined from the image and the position of the reference points can be corrected using the altitude angle. The altitude angle denotes the camera's upward or downward inclination at which the image of the person is taken. The screen is typically located in the straight viewing direction of the person while working on a PC; cameras on the PC therefore record the person typically from above since the camera is fastened to the upper edge of the screen. In the case of laptops, the camera typically sits below the straight viewing direction.

In order to determine the altitude angle, the camera can have a corresponding position sensor. If the camera is for example a smartphone camera, such a sensor is typically present since smartphones have in most cases already had a position sensor for years, which position sensor can ascertain the position of the device in space (relative to Earth's gravity) in order to suitably rotate the screen display for example.

If no such position sensor is present, the altitude angle can be determined from the image itself. For this purpose, for example, patterns for body contours that were taken from different altitude angles can be stored. These patterns are compared with the contours present in the image in order to determine the present altitude angle.

In some embodiments, straight lines present in the image can be determined. These often originate from edges of furniture or from the room itself in which the person is sitting and are vertical or horizontal with a greater likelihood. From their profile, it is also possible to estimate the angle at which the image is taken.

A further possibility for determining the altitude angle consists in using a neural network. This is expediently trained using images of people which are taken from different altitude angles and with different image portions. In this case, for simplification, the neural network can also be applied only to the contour of the person. The contour can for example by movement recognition.

Such determination does not have to be performed continuously since the altitude angle of the camera typically remains unchanged for a relatively long time. Substantial computing power can therefore also be used for the determination. The determination can for example also be carried out over a period of time using a plurality of images from the camera, for example a video portion, so that the determination does not fail due to problems of a single image (such as for example overexposure, underexposure, a sudden change in posture, error recognition in the case of a low-contrast image) or deliver false results.

The posture can be determined for a sequence of images from the camera and, out of the values determined in this way, those with a high probability of error are discarded. A high probability of error arises for example when successive values for the posture exhibit strong deviations from one another. This may indicate recognition of an error or a change in posture. A high probability of error also arises when the determined values leave an absolute expected range which defines anatomically likely postures. For example, it is unlikely that the ears are considerably below the shoulders. Moreover, a high probability of error can be assumed if the body reference points on both sides (left/right) are very unsymmetrical, that is to say for example cannot be substantially mapped onto one another by reflection at a possibly slanted axis.

Generally, even with values that seem correct in principle, the further processing can be delayed until a certain number of images, that is to say a certain video length, is evaluated, in order to guarantee high reliability of the evaluation. Since the evaluation does not rely on a high speed or frequency of the results, an interval of 10 s, 20 s or even 1 minute may well be used for this purpose. Since the individual images from the camera in a video recording common today in turn barely differ from one another, since they are recorded at a frequency of 40 ms for example, the evaluation of most of the occurring images can be dispensed with. For example, an image may only be evaluated at each interval of 1 s duration. In this case, the evaluation can consider a plurality of images and those for which the evaluation results in a high probability of error can be discarded.

Use can be made of at least two cameras, in particular a 3D-capable camera. The accuracy of the determination of the posture can be improved if spatial information instead of purely two-dimensional image information is available. Such information can determine the posture irrespective of sources of error such as spatial distortion and shortening. In this case, the cameras can be independent cameras, for example a laptop camera and an additional freely orientable camera. However, it may also be a matter of an integrated system for recording three-dimensional image information, that is to say a 3D camera which is likewise based on two cameras. For determining the three-dimensional position, it is advantageous if both cameras are positioned at a distance of a few cms with parallel viewing directions.

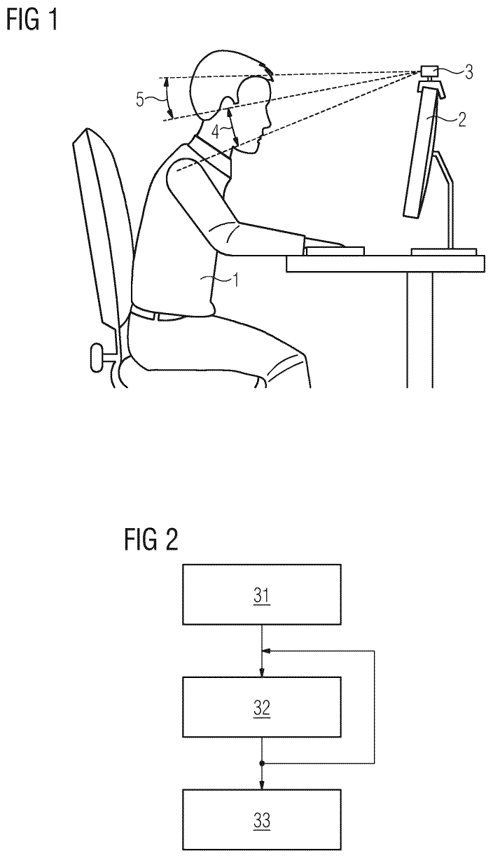

FIG. 1 shows a person 1 in a first posture while working on a PC not illustrated in FIG. 1. This first posture exhibits a posture that is bent forward, that is to say toward the PC screen 2. Such a posture hurts the back over the long term and is therefore disadvantageous.

FIG. 1 furthermore shows a camera 3 which is connected to the PC, is usually mounted on the top side of the screen and is oriented such that it captures the head and upper body of the person 1. The posture of the person may be determined by a program running on the PC. For this purpose, a method illustrated in FIG. 2 is used using a stream of images from the camera 3, that is to say its video output.

In a first step 31, the viewing angle at which the camera 3 is viewing the person 1 is defined. In this exemplary embodiment, the program identifies the apparent vertical distance 4 between the ears and the shoulder of the person 1. In this case, the viewing angle on this region, that is to say for example on the ears, is therefore attractive. The measure used can for example be the altitude angle 5. This is the angle between a horizontal viewing direction and the viewing direction to the ears.

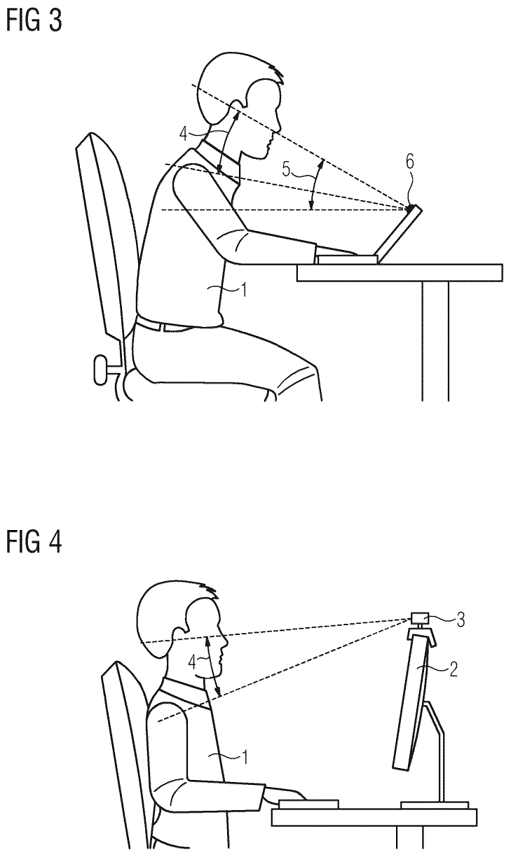

FIG. 3 shows an analogous view to FIG. 1, in which an integrated laptop camera 6 is used as the camera 3. As can be seen from FIG. 3, the altitude angle 5 here is a completely different one to the one for the camera 3 of FIG. 1.

The effect thereof can be seen by comparing with FIG. 4. FIG. 4 illustrates a straight and upright posture. The apparent distance 4 between ears and shoulders is, when the posture changes to that of FIGS. 1 and 3, smaller across the viewing angle of the camera for the camera 3 in FIG. 1, in an extreme case the distance could be zero, if the person leans far forward. In contrast, for the laptop camera 6, the distance across its viewing angle is actually a little larger.

The altitude angle 5 is determined by identifying straight running lines in the image and inferring the altitude angle 5 from the position of said straight running lines under the assumption that they are horizontal and vertical lines. For example, the table edges can be recognized or vertical lines of a door or room edge. If such lines cannot be recognized, the program can make provision for manual configuration which can be used to define the altitude angle 5.

In some embodiments, a 3D-capable camera can be used. In this case, spatial depth information is available, which information can be used for determining the posture. As a result, the dependency on the view angle is dispensed with and the determination of the altitude angle 5 can be dispensed with.

It is also possible, in addition or as an alternative to determining the altitude angle 5, to identify the position of body reference points directly by carrying out training with the person 1 beforehand. In this case, the person 1 can adopt different postures and the program records the arising positions of the body reference points. As a result, the dependency of the method on the present angles and corresponding calculations is smaller; the dependency on a direct input of the person 1 is greater for this, however.

In a second step 32, the ears and shoulders of the person are identified for a series of images from the camera. In this embodiment, identification is attempted on both sides. In this case, identification is carried out by a neural network which was trained beforehand on the basis of images of people.

In a third step 33, a probability of error for the results of the identification is determined. Included in this is whether identification has led to results which do not deviate too far from the results of previous images, provided that previous images are present. Furthermore included is whether the symmetry of ears and shoulders is within the realm of possibility and whether the arising posture is entirely almost vertical, that is to say is upright, or greatly tilted.

If too high a probability of error arises, which is checked on the basis of a threshold value, the result for this image is discarded and step 32 is returned to and the next following image is evaluated.

If the probability of error is small enough, the result of the evaluation is stored. Depending on the configuration, the evaluation can be continued for a particular number of images with step 32 until for example a defined number of images have been evaluated or a particular number of suitable results have been obtained.

If such a number is present, the determined posture is compared with a warning range in a step 33. In the simplest case, the determined distance 4 between ears and shoulders can be compared with a threshold value. In this case, the distance 4 has to be corrected for example for the altitude angle 5. If the threshold value is fallen below, the program can display a window which gives a warning. In this case, provision can also be made for such a warning to only appear when the posture is present for an extended period of time, for example 10 minutes.

### REFERENCE SIGNS

1 person
2 screen
3 camera
4 distance
altitude angle
6 laptop camera
31 first step
32 second step
33 third step

What is claimed is:

1. A method for monitoring a posture of a person while working on an electronic device, the method comprising:
reading an image from a camera associated with the electronic devices;
determining a value representing the posture shown in the image; and
transmitting a warning signal if the value falls in a warning range.

2. The method as claimed in claim 1, wherein determining the value includes analyzing relative apparent positions of body reference points in the image.

3. The method as claimed in claim 2, wherein the body reference points are selected from:
ears,
jaw,
shoulder, and
sternum.

4. The method as claimed in claim 2, wherein the relative apparent position comprises a vertical distance between the reference points.

5. The method as claimed in claim 2, further comprising determining the relative apparent position for two sides of the body; and
wherein identifying a deviating posture includes comparing the two sides.

6. The method as claimed in claim 2, further comprising:
determining an altitude angle at which the camera is recording the person from the image; and
correcting the position of the reference points using the altitude angle.

7. The method as claimed in claim 1, further comprising
determining the posture for a sequence of images from the camera; and
discarding any of images of the sequence having a high probability of error.

8. The method as claimed in claim 1, using at least two cameras.

9. An arrangement for determining the posture of a person working on an electronic device, the arrangement comprising:
a camera associated with the electronic device for image transmission purposes;
a program running on the electronic device to valuate an image received from the camera;
wherein the program:
reads an image from a camera associated with the electronic device;
determines a value representing the posture shown in the image; and
transmits a warning signal if the value falls in a warning range.
